(19) United States
(12) Patent Application Publication
(10) Pub. No.: US 2015/0111217 A1
(43) Pub. Date: Apr. 23, 2015

(54) MARKER QUANTITATION IN SINGLE CELLS IN TISSUE SECTIONS

(71) Applicant: MERRIMACK PHARMACEUTICALS, INC., Cambridge, MA (US)

(72) Inventors: Bart S. Hendriks, Cambridge, MA (US); Elena Geretti, Cambridge, MA (US); Arthur J. Kudla, Cambridge, MA (US); Matthew David Onsum, Cambridge, MA (US); Violette Paragas, Cambridge, MA (US); Sharon Moulis, Cambridge, MA (US)

(21) Appl. No.: 14/409,808

(22) PCT Filed: Jun. 20, 2013
(86) PCT No.: PCT/US2013/046914

§ 371 Date: Dec. 19, 2014

Related U.S. Application Data
(60) Provisional application No. 61/690,170, filed on Jun. 20, 2012.

(51) Int. Cl. G01N 33/569; G06T 7/00; G06K 9/00

(52) U.S. Cl. CPC G01N 33/56966; G06K 9/0014; G06T 7/0012; G01N 2333/71; G06T 2207/10056; G06T 2207/30096
USPC 435/6.19

(57) ABSTRACT

Improved assays incorporating single-cell based image analyses that enable quantitation of expression of individual cellular proteins and heterogeneity in terms of individual cellular protein molecule numbers per cell at the single cell level and mapped across sections of clinical tissue samples are disclosed.

### RELATED APPLICATIONS

This application claims the benefit of and priority to U.S. Provisional Patent Application No. 61/690,170, filed Jun. 20, 2012, the contents of which are incorporated herein by reference.

### BACKGROUND

Human epidermal growth factor receptor 2 (HER2 or ErbB2) is a cell surface protein that mediates signal transduction from extracellular stimuli into cells. HER2 overexpression, in which abnormally high levels of HER2 receptors are expressed on the surface of cells, occurs in multiple human cancers, and such abnormally high HER2 levels in tumor cells are associated with increased disease recurrence and poor prognosis. HER2 overexpression is often associated with HER2 gene amplification, a pathologic phenomenon associated with tumor cells in which a chromosomal region containing the HER2 gene is duplicated to yield multiple copies of the HER2 gene. Therapeutic agents targeted to HER2 include trastuzumab (Herceptin®), an anti-HER2 monoclonal antibody, and lapatinib (Tykerb®), a small molecule tyrosine kinase inhibitor which inhibits signal transduction by both HER2 and EGFR (HER1). These targeted agents have demonstrated clinical benefit in HER2-positive (i.e., HER2 overexpressing) breast and gastric cancer patients, particularly when combined with certain chemotherapies.

Current methods used to determine tumor HER2 status are sub-optimal, as they yield only a rough indication of the number of HER2 receptors expressed by each tumor cell and cannot determine if small subsets of cells within a tumor overexpress this receptor, nor do they provide for quantitative analysis of expression numbers across multiple tumor cells in sections of tumor. To select patients for HER2-directed therapy in the clinical setting, HER2 status is currently measured by immunohistochemistry (“IHC,” which measures protein levels) and/or fluorescent in situ hybridization (“FISH,” which detects HER2 gene amplification). These measures yield an imprecise prediction of response to HER2-targeted therapy. Previous work has described the development of automated quantitative analysis (AQUA) for measuring HER2 expression. AQUA utilizes a cytokeratin stain as a mask to identify tumor tissue followed by anti-HER2 staining and detection via immunofluorescence. Relative quantitation of HER2 expression is performed using automated image analysis based on cell line standards in a tissue-averaged manner, and heterogeneity is reduced to a single variable using Simpson's biodiversity index.

Existing IHC and FISH tests Two IHC tests for assessing HER2 status have been approved by the FDA: HercepTest® and Pathway (Ventana Medical Systems, Tucson, Ariz.). Both of these tests use immunohistochemical staining of the HER2 protein and are then interpreted by a pathologist who scores the degree of staining as 0, 1+, 2+ or 3+. Differences in sample handling, fixation, storage procedures and staining have all been shown to interfere with the antigen retrieval, stability and consequent reliability of IHC. In addition to issues of sample processing, the pathologist's interpretation can be subjective. Challenges in reproducibility across laboratories and pathologists, and even within pathologists themselves, further echo issues with the subjective nature of these tests. Specific training in one indication does not necessarily translate to others. The greatest need for attention in HER2 assessment is with regard to how 2+ samples are handled. The variation in the percent of samples scored as 2+ across multiple studies is nearly 5 times that of 3+ in both breast and gastric cancer and thus has a large impact on which patients are considered eligible for anti-HER2 therapy.

Three FISH tests have been approved by the FDA for assaying HER2 gene amplification: PathVysion® (Abbott, Abbott Park, Ill.), INFORM (VENTANA Medical Systems, Tucson, Ariz.), and PharmDx® (DAKO, Carpinteria, Calif.). HER2 gene amplification is assessed by counting fluorescent HER2 foci within the nuclei of at least 20 cells in two distinct pathologist-assessed tumor areas. In the PathVysion® test, centromere 17 (CEP 17) foci are also counted to report the ratio of HER2:CEP 17. This serves as an internal control, something lacking in IHC tests. Recent reports, however, are questioning the validity of using CEP 17 in conjunction with HER2. Polysomy of chromosome 17 has been shown to be a rare event, and it is likely that increased signals of centromere 17 are due to co-amplification with the HER2 gene. In these cases, patients would be incorrectly classified as non-amplified. According to the ASCO-CAP guidelines, more than 6 gene copies of HER2 per nucleus or a HER2:CEP 17 ratio greater than 2.2 is considered positive. Due to the counting of a small number of cells, FISH does not capture tumor heterogeneity. Further, recent analysis has suggested that looking at small numbers of cells can result in fluctuations that could influence inclusion criteria. One advantage of FISH over IHC-based tests is that FISH results are less sensitive to sample handling and processing since HER2 DNA is more stable than HER2 protein. In standard clinical practice, FISH interpretation still requires a pathologist. Attempts have been made to automate FISH interpretation, but they have not yet been adopted into standard clinical practice. FISH has a number of drawbacks compared to IHC based tests in that it is more expensive, it is technically more cumbersome and time consuming, and fewer laboratories have the ability to perform FISH. As a result, FISH is most commonly performed at centralized laboratories.

The extent to which tumor heterogeneity is prognostic or predictive of patient response to anti-HER2 therapy is unknown, and no FDA-approved test is currently able to report a quantitative measure of HER2 heterogeneity. With current testing methods it is not possible to systematically determine the optimal cutoffs for the percent of cells expressing a certain level of HER2 expression for optimal patient responses. In breast cancer, ASCO-CAP guidelines recommend intense staining of >30% of cells by IHC as a cutoff for positivity. By contrast, in gastric cancer the recommended cutoff is >10% of cells. Further, HER2 expression in gastric tumors shows considerable intratumoral heterogeneity, accounting for a large portion of testing discordance.

In HER2-positive metastatic breast cancer, response rates to trastuzumab-containing regimens vary from 36-79%. Furthermore, some HER2-negative patients respond to trastuzumab. Beyond breast and gastric cancers, there are other solid tumors such as certain bladder, endometrial and/or lung cancers that have been shown to overexpress HER2, and are not currently served by anti-HER2 therapy. There remain several challenges to accurate assessment of HER2 protein levels for patient stratification to distinguish responsive patient sub-populations from those that will not respond to HER2 targeted therapies. These include: intratumoral heterogeneity of HER2 expression and lack of high precision HER2 quantification techniques suitable for clinical use. There is also a need for quantitating tumor cell proteins other than HER2, so that tumors that do not overexpress HER2 can be analyzed and the results used to inform treatment decisions.

Thus, as current AQUA technologies do not provide for determining receptor expression numbers per cell or across the area of a two dimensional tumor section, there is a need for improved testing techniques for HER2 and other cellular proteins such as tumor-associated proteins to allow more precise and quantitative determination of protein expression levels at the cellular level and the distribution of protein expression levels in tumors so as to provide better data and criteria for distinguishing responsive patient sub-populations from those that will not respond to protein targeted therapies.

The present invention addresses these needs and provides other benefits.

### SUMMARY

Disclosed herein are improved assays incorporating single-cell based image analyses that enable quantitation of expression of individual cellular proteins (e.g., HER2) and heterogeneity in terms of individual cellular protein molecule numbers per cell at the single cell level and mapped across sections of clinical tissue samples (e.g., tumor samples).

In one aspect, a method is provided for quantitatively measuring levels of a cellular protein in each of a plurality of cells (e.g., target cells) in a section of a tissue sample so as to obtain an at least two dimensional (e.g., length and width) map of quantified density distribution of the cellular protein across the section, the method comprising (in order):
preparing a section from a tissue sample, said section comprising identifiable cells;
staining the section with a first stain specific to the cellular protein, a second stain specific to cell nuclei, and a third stain allowing the discrimination of target cells (e.g., malignant cells) from non-target (e.g., stromal) cells, wherein the first, second and third stains are distinguishable from each other when the stained section is imaged;
obtaining one or more microscopic images of the section wherein the first, second and third stains can be discriminated
identifying target cells within the one or more images based upon staining with the second and third stains;
measuring the intensity of staining with the first stain for a plurality of the identified target cells to obtain a plurality of cellular protein staining intensity data for individual cells and recording cell location coordinate data in association with cellular protein staining intensity data for each individual cell;
   ascertaining a level of stained cellular protein that is detected in each identified target cell by comparing stain intensity of the cellular protein in each identified target cell with stain intensity of the cellular protein in each of a plurality of standard cell preparations, the plurality including multiple standard cell preparations having differing known levels of expression of the cellular protein;
   creating a map of quantity distribution of the cellular protein in each of the target cells within a region of the section.

In one embodiment, the tissue sample is a tumor sample, e.g., a biopsy sample, the target cells are malignant cells, and the non-target cells are stromal cells.

In one embodiment, the cellular protein is a cell surface receptor. In one embodiment, the cell surface receptor is a growth factor receptor. In one embodiment, the growth factor receptor is an EGFR family receptor. In an exemplary embodiment, the EGFR family receptor is HER2. In other embodiments, the EGFR family member is HER3 or EGFR.

In one embodiment, the quantity distribution of the cellular protein is a continuous distribution.

In one embodiment, the first stain comprises an antibody specific to the cellular protein. In one embodiment, the second stain is a DNA stain. In another embodiment, the second stain comprises either or both of DAPI and a Hoechst® stain. A suitable Hoechst stain is, e.g., Hoechst33342 or Hoechst33258. In some embodiments, other DNA-staining molecules, such as doxorubicin, etc. may be used. In yet another embodiment, the third stain comprises an antibody. In one embodiment the antibody comprised by the third stain is specific to a cytokeratin.

In one embodiment, the map is in the form of a complementary cumulative distribution.

In another embodiment, the identifying and measuring and ascertaining are done by automated image analysis.

In one embodiment, the plurality of the identified target cells comprises at least 500 cells. In another embodiment, the plurality of the identified target cells comprises at least 1,000 cells. In another embodiment, the plurality of the identified target cells comprises at least 2,000 cells. In another embodiment, the plurality of standard cell preparations is in the form of an array of stained standard cells.

In one embodiment, the antibody is a labeled antibody. In another embodiment, the antibody is an unlabeled antibody that is subsequently labeled with a labeled secondary antibody specific to an antibody type characteristic of the first antibody.

### BRIEF DESCRIPTION OF THE DRAWINGS

FIG. 1 is a schematic overview of the assay. Cell lines with a range of HER2 expression as quantified by qFACS are used to generate a standard cell pellet array. The standard is stained in parallel with a tissue of unknown HER2 expression. Images of the standard and of the tissue are acquired and analyzed by automated image analysis tools. Analysis allows for the generation of a standard curve that can be used to interpolate HER2 numbers on a cell-by-cell basis on the tissue of unknown HER2 expression.

FIG. 2: (A) The cell pellet array was stained with an anti-HER2 and anti-cytokeratin antibody and counterstained with DAPI (4′,6-diamidino-2-phenylindole). The slide was scanned with an Aperio ScanScope FL® and analyzed with Definiens® Developer XD. The original and classification views of representative cores of the different cell lines of the cell pellet array are shown. The cytokeratin layer was omitted from the original views and only the HER2 (red) and DAPI (blue) layers are shown for simple visualization. In the classification views, HER2 low expressing cells (<˜150,000 HER2/cell) are shown in pink. Medium HER2-expressing cells (>˜150,000 and <˜1,000,000 HER2/cell) are shown in light red and high HER2 (>˜1,000,000 HER2/cell) are shown in red. HER2 negative cells are shown in grey. (B) Representation of the single-cell distribution of the LOG10 (Mean HER2 membrane intensity/cell) for the different cell lines of the standard. (C) The Mean HER2 membrane intensity/core of each cell line is plotted against the correspondent LOG10 HER2 receptor numbers, quantified by qFACS, to generate a standard curve. 95% interval of confidence is represented by the dashed lines. The curve was analyzed with a linear regression fit (R2=0.94). (D) The regression residuals (with 95% confidence intervals) are plotted for each cell line.

FIG. 3: (A) A breast disease TMA was stained with HER2 (red), cytokeratin (green) and DAPI (blue) and representative TMA cores at LOW (G1), MEDIUM (F8) and HIGH (D6) HER2 expression are shown (top panels). The corresponding cell segmentation and classification is shown in the bottom panels. Pink indicates LOW HER2 expression (<˜150,000 HER2/cell); light red indicates MEDIUM HER2 expression (>˜150,000 and <˜1,000,000 HER2/cell); red indicates HIGH HER2 expression (>˜1,000,000 HER2/cell), and cyan represents non-tumor cells/stroma. The Mean HER2 receptor numbers/cell, interpolated based on the standard cell pellet array stained in parallel with the TMA are shown in (B). (C) The distribution of HER2 expression among the different populations (HER2 HIGH, red; HER2 MEDIUM, light red; HER2 LOW, pink; and HER2 NEGATIVE, white) is shown for all the TMA cores.

FIG. 4: (A) Two consequent sections of the breast disease TMA were stained in parallel with a standard cell pellet array on two separate days. Mean HER2/cell on each individual core for the two different TMA sections were interpolated from the corresponding standard and plotted against each other. The data was fit in GraphPad Prism® with a linear regression giving an R2 of 0.98 and a slope=1.07. (B) Two standard pellet arrays (Standard A and Standard B) were stained on the same day. The Mean HER2 Intensity/cell membrane for the different cell lines of the standard were plotted against the correspondent LOG HER2 receptor number determined by qFACS. The standards were fitted in GraphPad Prism with a linear regression (R2 of 0.94 and 0.92 for Standards A and B, respectively). (C) A breast disease TMA was stained in parallel with the two above standard cell pellet arrays. Mean HER2 receptor numbers for the TMA cores were interpolated from either standard and were plotted against each other. The data was fitted in GraphPad Prism® with a linear regression (R2 of 1.00; slope=0.85).

FIG. 5: (A) The distribution of HER2 expression (HER2#/cell) in two representative breast carcinoma cores is shown. (B) The data in (A) was re-plotted using an inverse cumulative distribution. At the Y value of 0.5, 50% of the cells of the core represented in blue express more than 10,000 HER2/cell, and 50% of the cells of the core represented in red express more than 1,000,000 HER2/cell. (C) The Mean HER2 receptor numbers/core are plotted against the HercepTest® scores determined by a pathologist as 0/1+ (green), 2+(dark blue), and 3+ (red). (D) The HER2 expression on all the tumor cells of each individual core of the breast disease TMA is plotted as an inverse cumulative distribution and color-coded by the HercepTest® scores as in (C). (E) The Mean HER2 receptor numbers/core are plotted against the FISH scores obtained from staining of a nearby region tissue slide and scoring by a pathologist as FISH positive (POS, red), FISH negative (NEG, green) or borderline (blue). (F) The HER2 expression on all the tumor cells of each individual core of the breast disease TMA is plotted as an inverse cumulative distribution and color-coded by the FISH as in (E). Non-analyzable cores by either HercepTest® or FISH are indicated as dashed lines in both panel D and F. (G) The plot for each individual core was color-coded based on the traditional HER2 classification. The plots show that there is considerable variability of HER2 expression within any given sample. It is apparent that the “HER2-negative” patient samples have significantly fewer HER2 receptors per cell than the “HER2-positive” samples.

FIG. 6: Gastric, bladder and ovarian cancer TMAs were stained as described in the Examples in parallel with a standard cell pellet array. Slides were scanned with an Aperio Scanscope FL® and analyzed with Definiens® Developer XD. The original views of representative cores for the different tumor types are shown on the top panels (Her2, red; Cytokeratin, green; DAPI, blue). The corresponding classification views (HER2 HIGH, red; HER2 MEDIUM, light red; HER2 LOW, pink; non-tumor cell/stroma, cyan) as well as the inverse cumulative distribution functions are shown in the bottom panels.

FIG. 7: (A) A heart tissue microarray was stained with an anti-HER2 antibody and counterstained with DAPI. The slide was scanned with an Aperio ScanScope FL® and analyzed with Definiens® Developer XD. The original and classification views of cores of different heart conditions (normal and diseased) are shown. (B) The Mean HER2 membrane intensity/core for the different TMA cores is plotted. (C) The distribution of HER2 expression among the different populations HER2 HIGH (>˜1,000,000 HER2/cell, red), HER2 MEDIUM (>˜150,000 and <˜1,000,000 HER2/cell, light red), and HER2 LOW (<˜150,000 HER2/cell, pink) is shown.

FIG. 8: The histograms of HER2 expression for three sub-groups of HER2-positive samples are shown in (A), “HER2 low and heterogeneous”; these samples are HER2-positive in a clinical sense but have an overall lower expression and show heterogeneity with a dominant peak of lower-expressing cells, (B), “HER2 high and heterogeneous”; these samples are dominated by high HER2 expressing cells, but still have a significant amount of heterogeneity, and (C), “HER2 unambiguously high”; these samples show very high and uniform HER2 expression with little heterogeneity. The line colors in panels 8A-8C correspond to the IHC score for each sample (red=3+, dark blue=2+, and light blue=1+). Frequency represents probability density—the y-axis is normalized such that the area under the density curve is unity. The pie charts in (D) and (E) show the relative abundance of these sub-groups in the “IHC 3+, FISH+” and “IHC 2+, FISH+” categories, respectively (unambiguously high, dark red; high and heterogeneous, blue; low and heterogeneous, light green).

### DETAILED DESCRIPTION

Disclosed herein are methods for determining, at a single cell level, the amount of a cellular protein in a particular tissue type. The assay described below provides advantages by allowing objective quantitation of cellular proteins in terms of molecules per cell based on a fully characterized standard curve. The single cell-based analysis also allows for visualization of the heterogeneity of cell type in a sample, which has far-reaching therapeutic implications, for example, in tumor classification and treatment selection. In addition, the use of automated image analysis software in conjunction with the standard curve has the potential to minimize or possibly even remove reader subjectivity from the classification of cellular protein levels.

In an exemplary embodiment, the assay disclosed herein may be used to determine the level of a cellular protein in or on the cells in a tumor sample (e.g., a biopsy). A tumor sample suitable for testing by the assay may be, for example, from a tumor type associated with HER2 gene-amplified tumors and/or a HER2-expressing or overexpressing tumors. HER2 is a cell surface transmembrane receptor protein belonging to the ErbB family of receptors. HER2 (also referred to as ErbB2) generates intracellular signals (e.g., upon ligand activation of HER2 receptor that is dimerized with another ErbB receptor) via its intracellular tyrosine kinase activity. In excess, such signals can promote oncogenesis, e.g., by triggering cell division. The HER2 gene is amplified and/or overexpressed in many types of human malignancies, including but not limited to breast, ovarian, endometrial, pancreatic, colorectal, prostate, salivary gland, kidney, and lung. HER2 overexpressing cancers are designated a HER2+++ or HER2++ depending on the level of HER2 overexpression, with HER2+++ indicating the highest levels of HER2 expression. HER2+++ and HER2++ status are typically determined by an immunoassay such as HercepTest® (a semi-quantitative immunohistochemical assay for determination of HER2 protein overexpression). HER2 gene amplification may also be determined by, e.g., FISH (fluorescence in situ hybridization), with HER2-gene-amplified cancers being those that exhibit more than two HER2 gene copies per cell (typically two copies for every single copy of CEP17), and cells and/or tumors comprising HER2-gene-amplified cancer cells being referred to as “FISH positive.” In some embodiments a tumor sample may overexpress HER2 and yet not be FISH positive, e.g., a tumor sample may be HER2+++ or HER2++ (HER2 overexpressed at the protein level) but can be FISH negative (no detectable amplification of the HER2 gene).

The assay may be used to determine whether a cancer patient would respond to a targeted anti-cancer therapeutic, e.g., an antibody targeting at least one EGFR family member such as HER2, HER3, or EGFR. In one embodiment, the assay is useful to determine which patients that are HER2++ by HercepTest® have either high overexpression of HER2 in a subset of tumor cells, or a medium-to-high overexpression of HER2 on a large percentage of tumor cells. The assay may also be used to determine whether a patient should be treated with any ErbB-targeted anti-cancer therapeutic, e.g., trastuzumab, pertuzumab, lapatinib, MM-111, MM-121, MM-141, MM-151, or MM-302.

“MM-111” (also referred to as B2B3-1) is a bispecific HER2/HER3 antibody described, for example, in U.S. Patent Publication No. 2011-0059076 A1, and PCT Patent Publication number WO2009/126920. The HER2/HER3(ErbB2/ErbB3) oncogenic heterodimer is the most potent ErbB receptor pairing with respect to strength of interaction, impact on receptor tyrosine phosphorylation, and effects on downstream signaling through mitogen activated protein kinase and phosphoinositide-3 kinase pathways. HER3 signaling has been posited as an important mechanism of resistance to both HER2-targeted agents (such as trastuzumab) and chemotherapies (such as lapatinib) in clinical use. In HER2 high disease states one mechanism of activation of HER2 signaling is through binding of the ligand heregulin to a hetero-dimer of HER2 and HER3. Currently marketed HER2-targeted therapies do not effectively inhibit heregulin activated HER2/3. Preclinically, combinations of MM-111 (inhibiting heregulin activation of HER2/3 without blocking HER2) with trastuzumab (targeting HER2) provide complete inhibition of tumor growth.

MM-111 specifically targets the HER2/HER3 heterodimer and abrogates ligand binding. In preclinical models of HER2+ gastric, breast, ovarian and lung cancers, MM-111 inhibits ligand-induced HER3 phosphorylation, cell cycle progression, and tumor growth.

“MM-121” is a fully human monoclonal antibody that targets ErbB3, a cell surface receptor implicated in cancer. ErbB3 has been shown to be critical to the growth and survival of tumors, and the use of ErbB3 as a resistance mechanism by cancer cells is common across patient populations and tumor types. MM-121 is designed to inhibit cancer growth directly, restore sensitivity to drugs to which a tumor has become resistant, and delay the development of resistance by a tumor to other agents. MM-121 is described, e.g., in U.S. Pat. No. 7,846,440 and U.S. patent application Ser. Nos. 12/425,874 and 12/904,492 (“Antibodies against ErbB3 and uses thereof”).

“MM-141” is a fully human tetravalent antibody designed to target signaling of the P13K/AKT/mTOR pathway driven through IGF-1R and ErbB3 (HER3). PI3K/AKT/mTOR signaling is often activated in cancers in response to stress induced by chemotherapies or targeted anti-cancer medicines, and is believed to play a significant role in promoting tumor cell survival. MM-141 is described, e.g., in U.S. Patent Publication No. 2012-0269812 and U.S. patent application Ser. No. 13/778,984 (“Monospecific and Bispecific Anti-IGF-1R and Anti-ErbB3 Antibodies”).

“MM-151” is an oligoclonal therapeutic consisting of a mixture of three fully human monoclonal antibodies designed to bind to non-overlapping epitopes of the epidermal growth factor receptor, or EGFR. EGFR is also known as ErbB1. An oligoclonal therapeutic is a mixture of two or more distinct monoclonal antibodies. EGFR (ErbB1) has long been recognized as an important drug target in several malignancies, including lung, breast, colon, pancreatic and head and neck cancers. MM-151 is described, e.g., in PCT Patent Application No. PCT/US2012/04235 (“Antibodies Against Epidermal Growth Factor Receptor (EGFR) and Uses Thereof”).

“MM-302” refers to a HER2-targeted immunoliposome comprising an anthracycline anti-cancer therapeutic Immunoliposomes are antibody (typically antibody fragment) targeted liposomes that provide advantages over non-immunoliposomal preparations because they are selectively internalized by cells bearing cell surface antigens targeted by the antibody. Such antibodies and immunoliposomes are described, for example, in the following US patents and patent applications: U.S. Patent Publication No. 2010-0068255, U.S. Pat. Nos. 6,214,388, 7,135,177, and 7,507,407 (“Immunoliposomes that optimize internalization into target cells”); U.S. Pat. No. 6,210,707 (“Methods of forming protein-linked lipidic microparticles and compositions thereof”); U.S. Pat. No. 7,022,336 (“Methods for attaching protein to lipidic microparticles with high efficiency”) and U.S. Patent Publication No. 2008-0108135 and U.S. Pat. No. 7,244,826 (“Internal”).

Immunoliposomes targeting HER2 can be prepared in accordance with the foregoing patent disclosures. Such HER2 targeted immunoliposomes include MM-302, which comprises the F5 anti-HER2 antibody fragment and contains doxorubicin. MM-302 contains 45 copies of mammalian-derived F5-scFv (anti-HER2) per liposome.

In indications where an elevated HER2 level correlates with the assay may be beneficially used in indications such as bladder, endometrial or lung cancer, in which HER2 measurement has not yet been standardized. A correlation between HER2 amplification and disease stage was found in bladder cancer, where ˜14.2% of grade 3 tumors (vs. 1.1% of grade 1 tumors) showed amplification. Bladder cancer is particularly interesting because it appears to be a cancer type where over-expression of the HER2 protein is not always accompanied by gene amplification. Such cases might be particularly well-suited for treatment with HER2-directed therapies that do not rely on addiction to HER2 signaling for their mechanism of action, such as HER2-targeted liposomal doxorubicin (e.g., MM-302) or anti-HER2/HER3 bispecific antibodies (e.g., MM-111).

According to the methods disclosed in the Examples below, quantitation (absolute or relative) of a cellular protein from a tissue section requires the generation of a standard curve that relates tissue staining to cellular protein levels. The standard curve is generated by measuring cellular protein expression levels in a panel of cell lines, and then by creating an array from pellets of these cells to be stained in parallel with the tissue sample of interest. In some embodiments, a standard curve generated by measuring cell free protein standards, e.g., protein spots of known concentration on a substrate, may be similarly employed.

### EXAMPLES

Disclosed herein is the development and application of a quantitative immunofluorescence method for determining HER2 protein expression at the single cell level in formaldehyde-fixed, paraffin-embedded (FFPE) tissue samples. The two key aspects of this assay that define and differentiate it from previous work, are (i) the ability to quantitate HER2 staining at the single cell level through the use of automated image analysis software that segments individual cells and (ii) objective quantitation of HER2 in terms of molecules per cell based on a fully characterized standard curve.

Materials and Methods

Materials—RPMI, Leibovitz's L-15 Medium, and McCoy's 5a Medium Modified is from LONZA (Walkersville, Md.), Fetal Bovine Serum (FBS) is from Tissue Culture Biologicals (Seal Beach, Calif.) and penicillin G/streptomycin sulphate mixture was from GIBCO (Invitrogen, Grand Island, N.Y.). Peroxidazed 1, Background Sniper and Da Vinci Green were from Biocare Medical (Concord, Calif.). Mouse anti-human pan cytokeratin and Envision anti-rabbit HRP were from Dako Cytomation (Carpinteria, Calif.). Rabbit anti-human HER2 (clone SP3), TRIS-EDTA buffer (100×) and TBST buffer (20×) were from Fisher Scientific (Pittsburgh, Pa.). TSA™ Cyanine 5 Tyramide Reagent was purchased from PerkinElmer Life Sciences (Waltham, Mass.). Goat anti-mouse Alexa555 and ProLong® Gold with DAPI were from Invitrogen (Carlsbad, Calif.).

Tissue culture—DU145, MDA-MB-175-VII, MDA-MB-453, ACHN, and SKOV3 cells were obtained from ATCC and grown under recommended conditions. IGROV1 were from NCI-DTP. BT474-M3 cells are a cell line highly overexpressing HER2 derived from BT474 cells (ATCC HTB-20®). MCF-7 clone 18 cells are a gift from Dr. Christopher Benz (Buck Institute, Novato, Calif.).

HER2 quantification in cell lines—Cells were trypsinized, washed and stained using fluorescently-labelled trastuzumab. Trastuzumab was labeled as previously described (Schoeberl B, Sci Signal 2009). HER2 receptor numbers were determined by assessing the antibody binding capacity (ABC) of the fluorescently-labeled HER2 antibody via quantitative fluorescence activated cell sorting (qFACS). ABC was determined using Simply Cellular Quantum Beads (Bangs Labs, Fishers, Ind.) per the manufacturer's instructions.

Cell pellet array—For each cell line, 2.5×108 cells at 80% confluence were rinsed with PBS and covered with 10% neutral buffered formalin at RT for 10 min with gentle agitation. Cells were collected by scraping, pelleted at 1000 rpm, 10 min at 4 C and re-suspended in 70% ethanol. Cells were pelleted and transferred into an Eppendorf tube prepared with a bed of paraffin. Cells were packed by centrifuging at 12,000 rpm for 5 min at RT followed by aspiration of the ethanol. Cell pellet molds were prepared by placing Eppendorf tubes in an embedding mold and surrounding with molten (55° C.) 1% low-melt agarose in TBS and allowed to set. Cell pellets were placed in the center of the agarose mold and sealed with agarose. Agarose-embedded cell pellets were immersed in 70% ethanol at 4 C until being embedded in paraffin and sectioned (Mass Histology Service, Inc., Worcester, Mass.).

Patient samples—Five micron sections of a breast disease spectrum tissue microarray (TMA) were obtained from Folio Biosciences (Powell, Ohio). Duplicate 1 mm tissue cores from 48 patients were represented on the TMA. Heart TMAs were from US Biomax (Rockville, Md.).

Immunofluorescence staining & image acquisition—The cell pellet array and a breast cancer TMA are stained with an anti-human pan cytokeratin antibody and an anti-human HER2 antibody as follows. Slides are baked for 30 min at 65° C. and de-paraffinized by immersion in xylene (2×30 min), 100% Ethanol (2×2 min), 80% Ethanol (2×2 min), followed by water. Antigen retrieval was accomplished by heating the slides in TRIS-EDTA buffer, pH 9, for 25 min at 95 C in a pre-treatment module (Thermo Scientific, Waltham, Mass.). After antigen retrieval, slides were stained on a Lab Vision Autostainer® 360 (Thermo Scientific). Briefly, endogenous peroxidase activity was blocked with Peroxidazed® 1 (10 min at RT) followed by a washing step with TBST and a protein blocking step with Background Sniper® (10 min at RT). Next, slides were incubated with the mouse anti-human pan cytokeratin and rabbit anti-human HER2 antibodies diluted in Da Vinci Green for 1 hr at RT. After washing, slides were incubated with a goat anti-mouse Alexa555 antibody diluted in Envision anti-rabbit HRP for 30 min at RT. After washing, incubation with TSA™ Cyanine 5 Tyramide Reagent for 5 min at RT followed. Slides are washed and mounted with ProLong® Gold mounting media with DAPI. For the quantification of HER2 on human heart tissue specimens, a heart TMA containing both normal and diseased tissues, and a cell pellet array were stained as above, with the omission of the cytokeratin antibody.

Cell pellet arrays and TMAs were scanned on a fluorescent ScanScope FL® (Aperio, Vista, Calif.) at a 20× magnification with a 0.75 Plan Apo objective.

Automated image analysis—automated image analysis was performed using custom rulesets written in Definiens® Developer XD (DEFINIENS, Munich, Germany). Briefly, nuclei were segmented in the DAPI layer. Subsequently, cells were identified by growing the nuclei until reaching the edge of the cytokeratin signal. The cytokeratin signal was used to distinguish between tumor cells (cytokeratin positive) and non-tumor cells/stroma (cytokeratin negative). The intensity of the HER2 membrane staining was quantified on a single-cell basis as the (mean of the inner border of the HER2 layer)+(mean of the outer border of the HER2 layer). For the quantification of HER2 on heart tissue samples, and relative standard pellet array, a modification of the above analysis was used in that, after nuclei detection, cells were outgrown until reaching the HER2 membrane staining. The intensity of the HER2 staining was quantified and used to classify cells into HER2 positive and negative cells. In the case of the cell pellet array, the values of the mean HER2 membrane intensities of the cores of the different cell lines were exported and plotted against the corresponding log (HER2 receptor numbers) determined by qFACS to generate a standard curve. In the case of the TMAs, the HER2 membrane staining intensity values of each single tumor cell of the core was exported and further analyzed based on the generated standard. Rulesets are available upon request.

HER2 IHC testing—Patient tumor samples were tested with HercepTest® (DAKO, Carpinteria, Calif.) according to the manufacturer's directions, performed by Folio Biosciences (Powell, Ohio). The TMA was scored using the ASCO/CAP guidelines for HercepTest® interpretation.

HER2 FISH testing—FISH analysis was carried out at the Dana-Farber/Harvard Cancer Center (DF/HCC) Cytogenetics Core Facility (Brigham & Women's Hospital Boston, Mass., USA). A breast cancer TMA was hybridized with a two-color commercial FISH probe (PathVysion® HER2 DNA Probe Kit, Abbott Molecular) containing the HER2/neu region (SpectrumOrange), and a chromosome 17 enumeration probe, CEP 17 (SpectrumGreen). Control slides (Abbott Molecular) were run concurrently with the breast cancer TMA. The assay was performed according to the manufacturer instructions. Stained slides were imaged on an Olympus BX51 microscope, using a CCD camera (ER3339) and the CytoVision® 3.6 Build 16 imaging software, both supplied by Applied Imaging Corp. The TMA cores were initially imaged in the DAPI channel at low power through a 10× objective, to identify the tumor areas. Subsequently, actual scoring was accomplished by imaging through a 100× oil objective. According to the Abbott guidelines and consistent with ASCO/CAP guideline, analysis of the TMA cores was carried-out by scoring a minimum of 20 identifiable tumor cell nuclei, or if no obvious tumor was identified after scanning the entire core, 20 ductal cells were scored. If neither tumor nor ductal cells could be identified, the core was considered non-analyzable (NA). Cores with a HER2:CEP 17 ratio of <1.8 were considered non-amplified, and those with a HER2:CEP 17 ratio of >2.2 were considered amplified. Cores with a HER2: CEP 17 ratio between 1.8 and 2.2 were considered borderline amplified, and additional cells were scored. Cores with no visible signal in one or both hybridization colors were considered not analyzable (NA).

Data analysis—Output from the automated image analysis of patient samples and cell pellet arrays (standards) were analyzed using MATLAB R2011a (The MathWorks, Natick, Mass.). Calibration curves of mean fluoresce intensity (MEI) to log-transformed HER2 receptor number were generated using linear regression on the quantified images from the cell pellet array. For each TMA, a cell pellet array was stained in parallel and the resulting calibration curve was unique to that TMA. HER2 membrane staining intensities on a per cell basis from the TMAs were interpolated based on the calibration curve from the corresponding cell pellet array. The distribution of HER2 receptor numbers for the tumor cells in a TMA core was represented with the complementary cumulative distribution (or “tail distribution”). This representation facilitated the identification of the percentage of cells within a core that exceeded a given HER2 receptor value.

### Example 1

### Assay Development

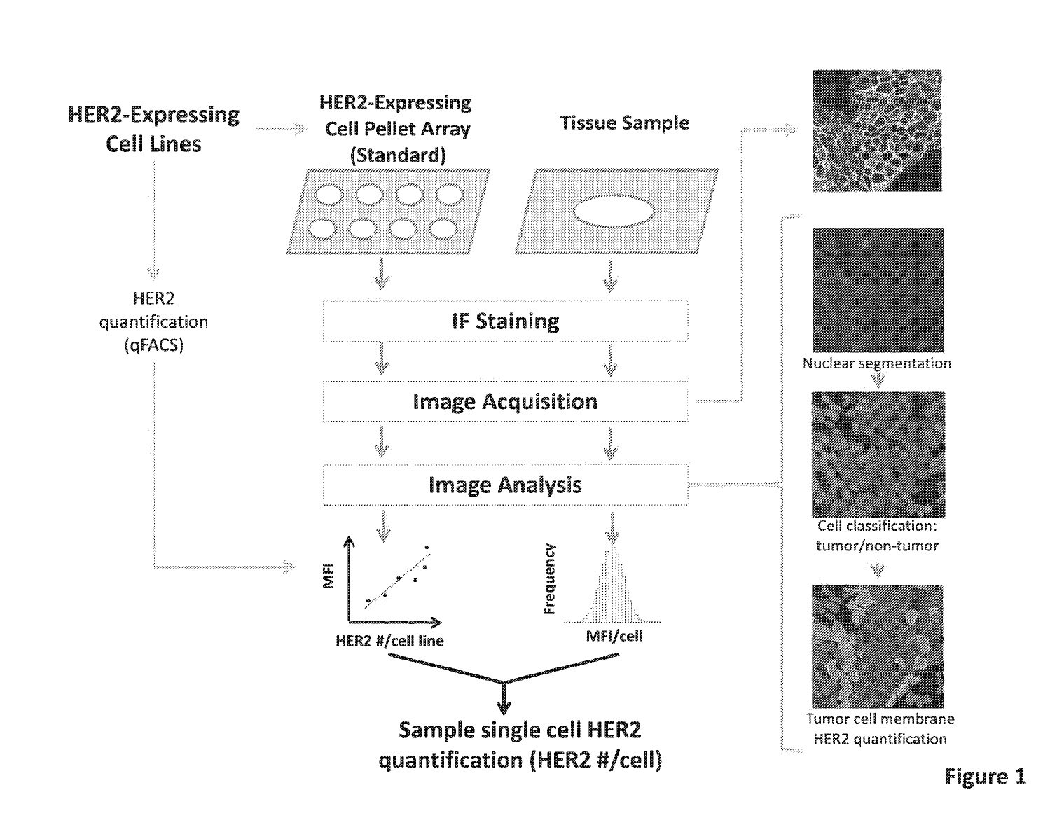

The use of automated image analysis enables the evaluation of larger sections of tumor and this will allow for a more accurate assessment of HER2 expression level, which may result in improved clinical benefit. Since anti-HER2 therapeutics act at the protein level, an assay was designed for protein detection, and coupled with a quantitative and objective analysis method. A high-level overview of the assay is shown in FIG. 1 and is described in detail below.

Quantification at the single cell level will be critical for assessing the impact of the heterogeneity of HER2 expression within a tumor on patient outcome, something that is not possible with current clinical HER2 assays. Further, if used retrospectively, the assay could objectively determine the optimal degree of HER2 expression and percent of HER2-positive cells to use as a diagnostic cut-point for prescribing HER2-directed therapies; this cut-point is actively being debated in both breast and gastric cancer but cannot be determined satisfactorily using currently available assays. In addition, the use of automated image analysis software and a standard curve has the potential to remove reader subjectivity from the classification of HER2 status.

Cell Pellet Microarray Generation. Quantitation of HER2 from a tissue section required the generation of a standard curve that related tissue staining to HER2 receptor levels. This standard curve was generated by measuring HER2 expression levels in a panel of cell lines and then an array was created from pellets of these cells to be stained in parallel with the tissue sample of interest. Eight cell lines (ACHN, DU145, IGROV1, MDA-MB-175-VII, MDA-MB-453, MCF7-clone 18, SKOV3, BT474-M3) were selected to span a wide range of HER2 expression, as measured by qFACS (Table 1). All cell lines included in the cell pellet array had a single HER2 population, as evaluated by FACS. Cores from each of the cell line-derived cell pellets were placed on the array in quadruplicate. The completed cell pellet microarray was sectioned and stained in parallel with tissue samples of unknown HER2 expression levels.

TABLE 1
Cell lines selected for the standard cell pellet array and
correspondent HER2 receptor numbers as determined by qFACS:

| | Mean |
|---|---|
| Cell line | Her2 (#/cell) |
| CHO-K1 | 3,000 |
| ACHN | 45,000 |
| Du145 | 69,000 |
| IGROV-1 | 158,000 |
| MDA-MB-175-VII | 202,000 |
| MDA-MB-453 | 393,000 |
| MCF-7 cl18 | 1,030,000 |
| SKOV-3 | 1,380,000 |
| BT474-M3 | 1,940,000 |

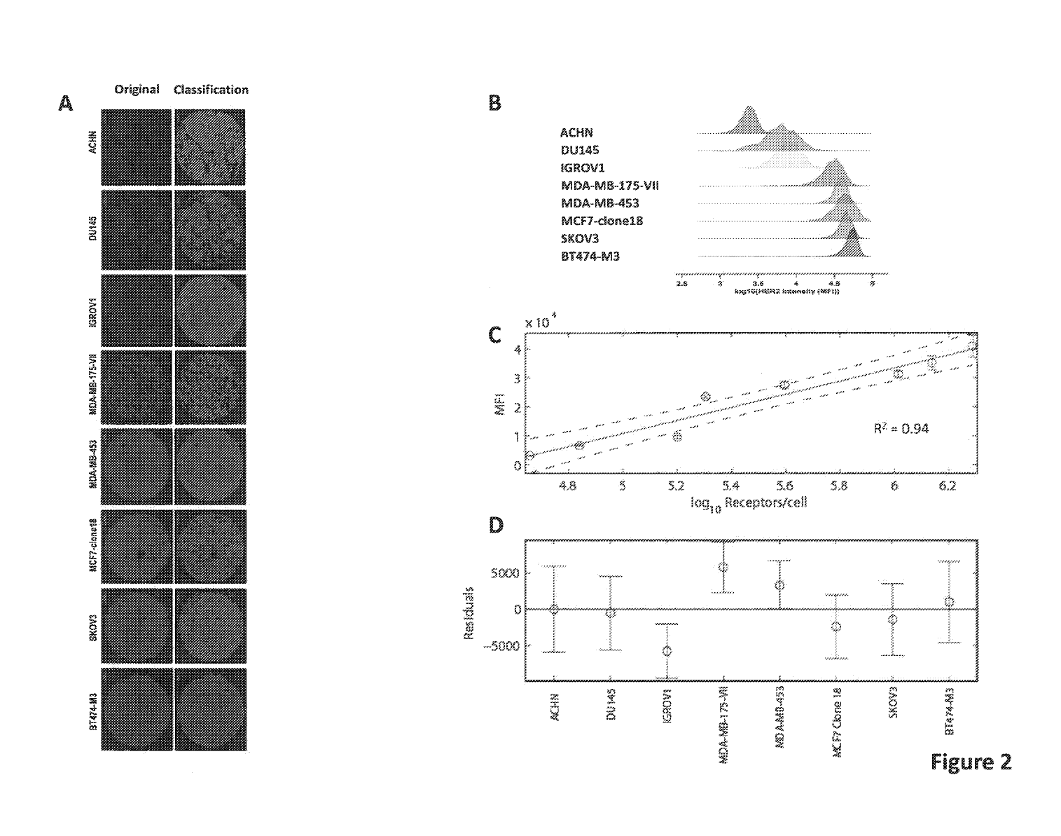

Staining and Image Analysis. The cell pellet array standard and tissue microarrays of unknown HER2 levels were stained in parallel to ensure consistency of staining. The staining was performed as described above with an anti-human pan cytokeratin antibody to distinguish tumor from non-tumor cells, an anti-human HER2 antibody to identify HER2, and counterstained with DAPI to identify cell nuclei. Images of entire sections of the cell pellet array were digitally acquired for subsequent analysis. Representative images of the cell pellet array cores of the different cell lines are shown in FIG. 2A (left panels).

After image acquisition, automated image analysis software quantified the HER2 staining of each cell in the cell pellet array and of the tissue microarray. The same image analysis algorithm was applied to both the cell pellet standards and the tissue microarray. The analysis consisted of (1) cell segmentation, (2) identification of tumor and non-tumor cells based on the cytokeratin stain, and (3) quantification of HER2 staining along the membrane on a cell-by-cell basis for all the identified tumor cells. The algorithm was designed in such a manner to obviate the need for user input and ensure fully objective operation. Segmentation and classification of the cell pellet microarray is shown in FIG. 2A (right panels). The distribution of the HER2 membrane staining intensity per cell for all the cell lines of the standard array is shown in FIG. 2B and indicates single populations of HER2 expression, consistent with observations by qFACS.

Assay Qualification

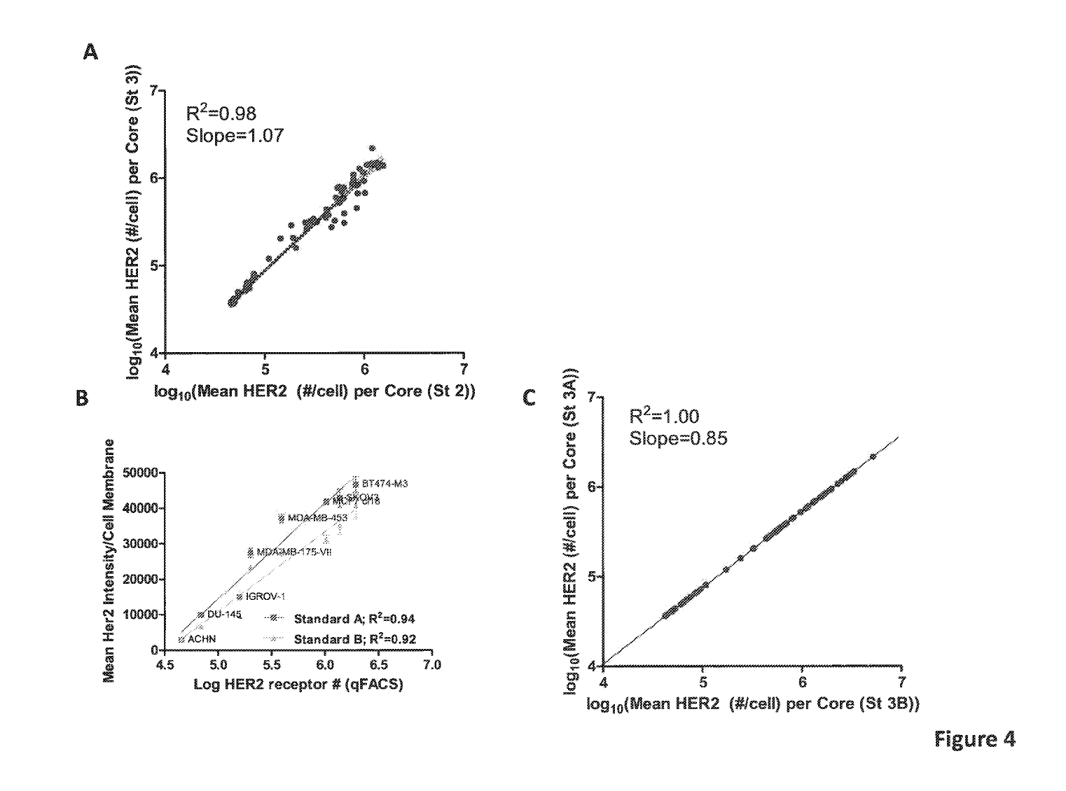

Standard Curve. Image analysis of the cell pellet array enables calculation of the average HER2 mean fluorescence intensities (MFI) for each cell line. These values were combined with qFACS measurements to generate a standard curve of MEI vs. HER2 expression in terms of receptors per cell (FIG. 2C). Based on goodness of fit and an analysis of residuals, it was determined that a log-linear calibration model gave the most robust and parsimonious fit to the data (FIG. 2D), yielding a measurement accuracy of +/−50,000 receptors/cell. Based on the standard curve, the lower and upper limit of quantitation is roughly 4.8e4 and 1.9e6 receptors/cell, respectively. The lower and upper limits of detection vary slightly from staining run to staining run. Staining of the standard curve and correspondent analysis was run on multiple days (n=8) and no significant differences were observed in the resulting standard curve, indicating there is negligible run-to-run error in this assay (also see FIG. 4).

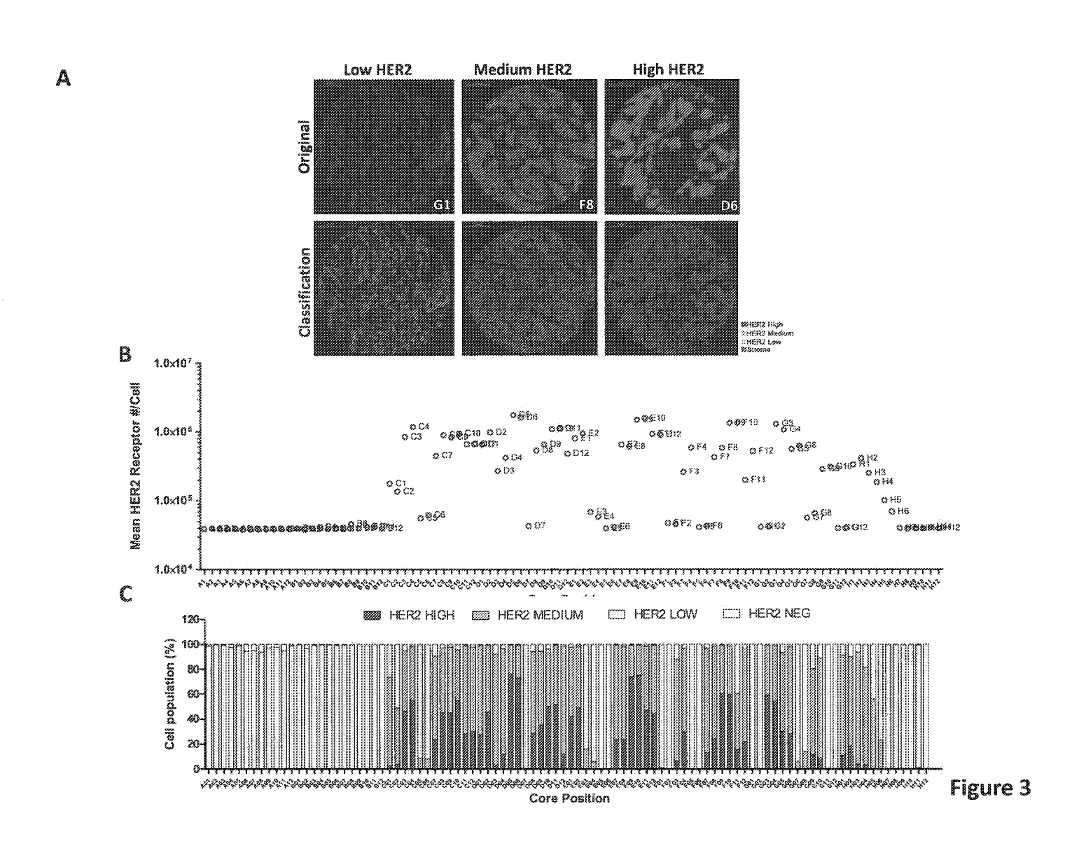

Sample Analysis. A breast disease TMA with 48 patient samples measured in duplicate (total of 96 cores) that included different stages of breast cancer and breast cancer types, as well as normal breast tissue (as control), was stained for HER2, cytokeratin and counterstained with DAPI. Staining of the breast disease TMA was run in parallel with the standard cell pellet array. Three tumor cores are shown in FIG. 3A (top panels). The corresponding cell segmentation by automated image analysis is also shown (bottom panels). HER2 negative tumor cells are shown in grey and tumor cells with low HER2 expression (<˜150,000 HER2/cell) are shown in pink. Medium (>˜150,000 and <˜1,000,000 HER2/cell) and high (>˜1,000,000 HER2/cell) HER2-expressing cells are indicated in light red and red, respectively. Cytokeratin-negative cells were classified as non-tumor/stroma cells and are represented in cyan. Using the standard curve, mean HER2 expression numbers per cell were calculated for each individual core (FIG. 3B). The percentages of the different tumor cell populations (HER2 low, pink; medium, light red; high, red; and negative, white) for each of the breast disease TMA cores are shown in FIG. 3C.

Assay Reproducibility. We assessed the technical reproducibility of our assay by comparing the mean HER2/cell values determined from near-identical sections on the breast tumor TMA described above. Consequent sections of the tumor microarray were stained in parallel with a control array standard on different days and the individual HER2/cell levels for the individual cores of the array were determined. Mean HER2/cell values for each of 96 cores on the tumor microarray from the two sections are shown in FIG. 4A to aid in visual comparison. The overall concordance is excellent, with an R2 of 0.98 and a slope=1.07.

The effect of the standard curve on the calculated HER2/cell values was investigated by staining two control arrays in parallel with a single breast cancer TMA. Shown in FIG. 4B are the two standard curves obtained from the staining of two control arrays (Standard A and Standard B). After simultaneous interpolation of the mean HER2/cell for each of the 96 individual cores from either Standard A or Standard B, a correlation plot was generated and is shown in FIG. 4C. A high overall concordance between the two interpolations was observed (R2=1.00; slope=0.85). From these data we can estimate the uncertainty in mean HER2/cell derived from variability in cell standards to be roughly 15%, a highly accurate measurement considering that HER2 protein expression ranges across 3-4 logs.

### Example 2

### Single-Cell Analysis of Patient Samples

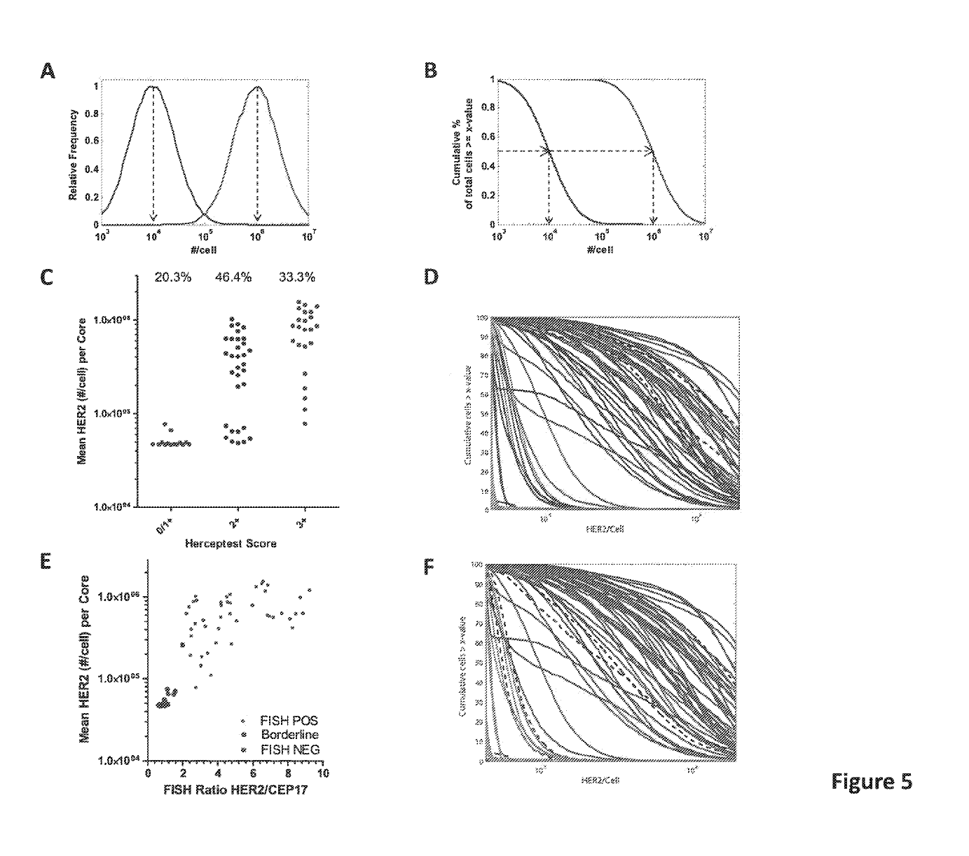

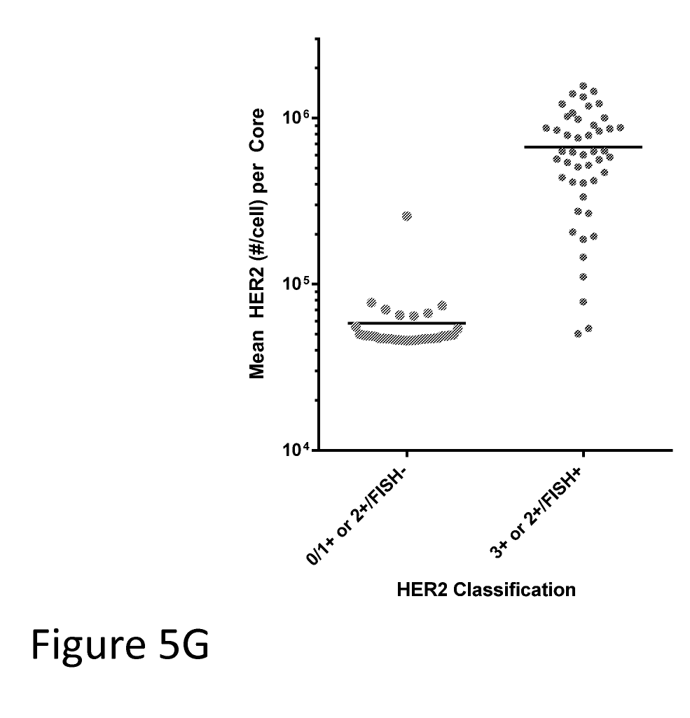

Breast Disease Samples. A broad disease spectrum TMA was utilized for analytical validation of our assay. The TMA contains samples from normal breast and a variety of stages of breast cancer. Consequently, it does not capture the typical distribution of HER2 positivity, either in terms of HercepTest® and/or FISH, as reported by broad-based surveys of HER2 positivity. The breast TMA was stained and analyzed in parallel with a cell pellet array and the HER2 level/cell in each core of the TMA was back calculated from the log-linear calibration curve. Representative histograms of the distribution of HER2 expression for two TMA cores are shown in FIG. 5A. Using these distributions, we can re-plot the data as an inverse cumulative distribution function to highlight the fraction of cells expressing greater than a given HER2 level (FIG. 5B). Plotted in this manner, our assay is able to quantitatively deliver two key measurements—HER2 expression/cell and the percentage of cells expressing the indicated level of HER2.

Comparison with HercepTest®. The mean HER2 receptor numbers per core were plotted against the HercepTest® scores determined by the TMA manufacturer in a nearby region tissue slice (FIG. 5C). An overall correlation between high HER2 receptor numbers and high (3+) scores was observed. However, it was also noticed that cores scored for a particular value by HercepTest® span over a wide variety of interpolated mean HER2 receptor numbers. As a next step of the analysis, instead of focusing on the mean HER2 numbers/core, which do not represent well the heterogeneity of the tissues, the distribution of HER2 expression among all the tumor cells in each of the TMA cores, was analyzed through an inverse cumulative distribution function as described in panels 5A and 5B. The obtained plot for each individual core was color-coded based on the correspondent HercepTest® score for that particular TMA core (3+, red; 2+, dark blue; 1+/0, green). The results are shown in FIG. 5D. The plots show that there is considerable variability of HER2 expression within any given sample. It is apparent that the samples segregate into two distinct populations, one on the left side of the graph, with 90% of the tumor cells/core expressing less than 100,000 HER2/cell and that includes most of the HercepTest® negative, 1+ cores and a few 2+ cores; and one population on the right side of the graph, with at least 30% or more of the tumor cells/core expressing at least 400,000 HER2/cell. This right-hand side population includes the majority of the HercepTest 3+ and 2+ cores.

Comparison with FISH. The results of the improved assay were also compared with FISH testing for HER2 amplification using PathVysion®. FISH was performed on a nearby tissue slice of the same breast disease TMA. HER2 amplification, as measured by the ratio of HER2:CEP 17 for each individual core is plotted against the corresponding interpolated mean HER2 receptor numbers per core in FIG. 5E. Cores with a high level of amplification were also characterized by high mean HER2 receptor numbers as determined by our assay, but this correlation did not hold for all the FISH amplified cores. When the distribution of HER2 expression among all the tumor cells in the core was analyzed through an inverse cumulative distribution function, again the cores clustered into two distinct populations (FIG. 5F). Cores with high percentages of tumor cells expressing high HER2 levels clustered on the right side of the graph and were all FISH amplified (red). Conversely, the FISH non-amplified cores (green) clustered to the left side of the graph, and had low % of tumor cells expressing high HER2 levels. Following the clinical HER2 classification schema, individual samples within the TMA were separated into two groups—(i) those that would not be eligible for anti-HER2 therapy (0/1+/2+ & FISH-negative; “HER2-negative”) and (ii) those that would be eligible for anti-HER2 therapy (2+ & FISH-positive and 3+; “HER2-positive”). The mean HER2 receptor numbers per core are plotted against these traditional definitions of HER2 shown in FIG. 5G. From the analysis of receptor numbers, there is a clear distinction between the two groups, based on the combination of HercepTest and FISH testing. In summary, the data show high concordance between our assay and FISH amplification.

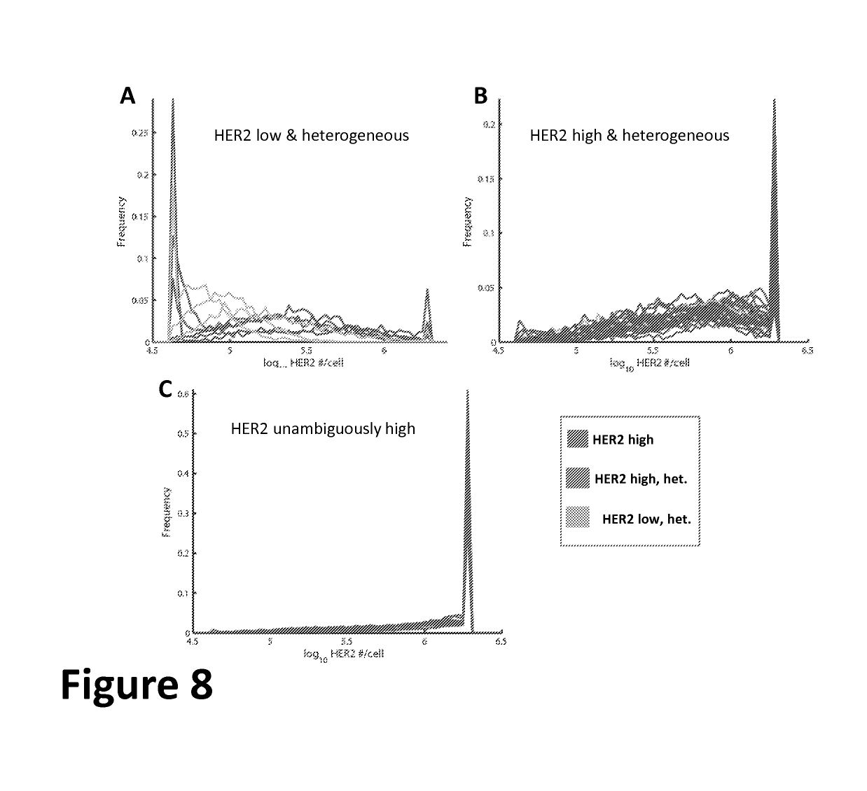

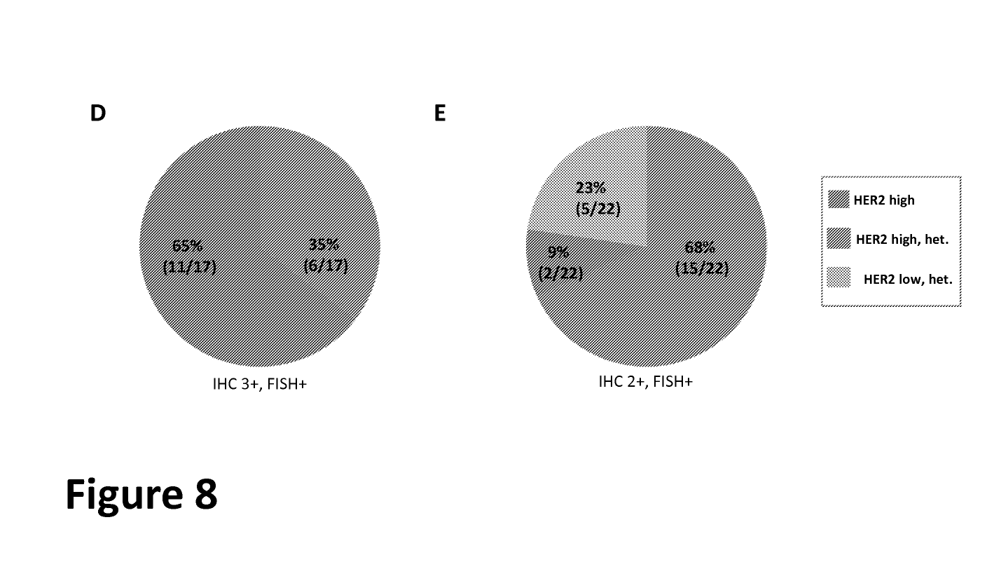

Clustering analysis. The HER2 quantitation, visualized using the inverse cumulative distribution function in FIG. 5, indicated that there was substantial heterogeneity in the distribution of HER2 in the patient samples, particularly within the “HER2-positive” group. Since individual patient response to therapy within this group is variable, distinct sub-groups within the “HER2-positive” group were identified and compared to results from traditional testing methods. HER2 receptor distributions for each patient tumor core in the HER2-positive group were clustered using the K-means algorithm. This analysis identified three distinct sub-groups in the traditional “HER2-positive” group, shown in FIG. 8A-C. The line colors in FIGS. 8A-8C correspond to the IHC score for each sample. Visual inspection of these sub-groups revealed three patterns: (1) low and heterogeneous—samples that had heterogeneous expression dominated by comparatively lower HER2 expression, but still classified as HER2-positive by traditional means (FIG. 8A), (2) high and heterogeneous—samples exhibiting variable degrees of intermediate expression, but dominated by high HER2 expression (FIG. 8B) and (3) unambiguously high—samples with a vast majority of cells expressing high levels of HER2 (FIG. 8C). All three subgroups show greater expression of HER2 than the traditional “HER2-negative” group. In FIGS. 8D and 8E, the proportion of each of the defined sub-groups is shown within either the HercepTest 3+ samples or the 2+/FISH-positive samples. The 3+ patient samples were 82% (14/17) unambiguously high and the remaining 18% (3/17) high and heterogeneous (FIG. 8D). By contrast, the 2+/FISH-positive patients exhibited a broader distribution with only 23% (5/22) unambiguously high, 54% (12/22) high and heterogeneous and the remaining 23% (5/22) low and heterogeneous (FIG. 8E).

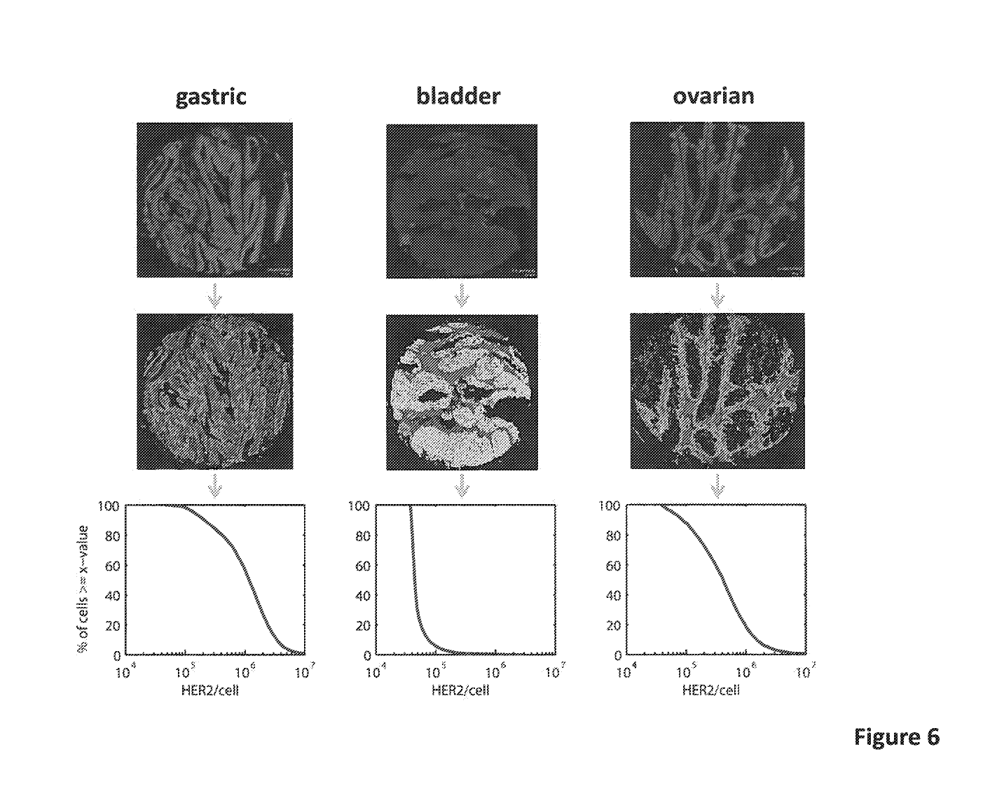

Other Tumor Types. Since HER2 testing is already well-established in the clinic for both breast and gastric cancer and the use of HER2-targeted therapies in additional cancers is being investigated, it is important the improved assay have applicability across a wide range of tumor types. To test the robustness of the assay and image analysis methodology, tumors of gastric, bladder and ovarian origin were stained, classified and scored. Stained images of representative gastric, bladder and ovarian tumor cores are shown in FIG. 6, along with their corresponding classification of tumor vs. non-tumor cells and the intensity of HER2 staining. The correspondent inverse cumulative distribution functions for the represented cores are shown in the bottom panels. The distinctly different morphologies of the different tumors are adeptly handled by the analysis method disclosed herein.

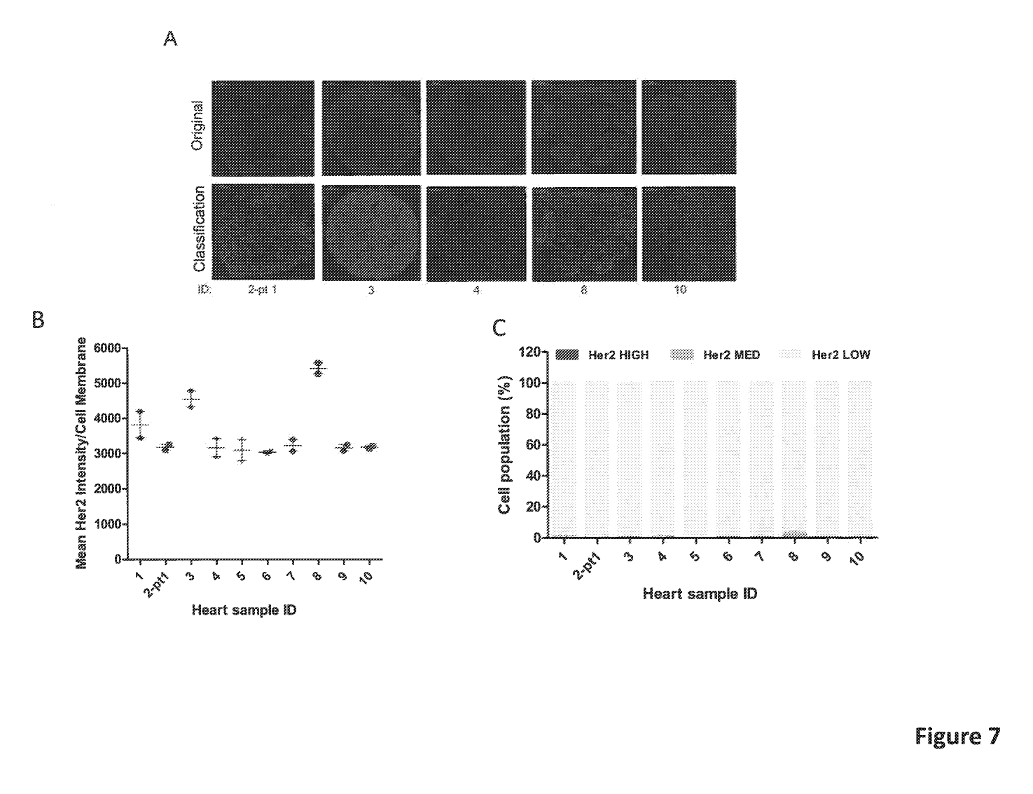

Human Heart Samples. To demonstrate the applicability of the assay on normal tissue, human heart tissue was examined. Beside its role in tumor progression, HER2 has been shown to also have a protective role for cardiomyocytes exposed to stress. It has been previously shown that human stem cell-derived cardiomyocytes express low levels of HER2 in vitro. A heart tissue microarray, including both normal and diseased heart specimens (the pathology diagnosis is shown in Table 2), was stained for HER2 and counterstained with DAPI in parallel with the above described cell pellet array standard. Representative images of the stained cores are shown in FIG. 7A (top panels). The heart TMA and cell pellet array were analyzed as described above, in a fashion similar to the paired breast cancer TMA and pellet array described above. The results of the segmentation and classification of representative heart cores are shown in FIG. 7A (bottom panels). The mean HER2 intensity/cell membrane for each of the heart cores analyzed is represented in FIG. 7B and the distribution of the different HER2 cell populations (HIGH, black; MEDIUM, gray; and LOW, light gray) is shown in FIG. 7C. All the heart samples showed low mean HER2 intensity levels, in the same range of the lowest HER2-expressing cell line (ACHN, 45,000 HER2/cell, Table 1). Over 95% of the cells in the core were classified as low HER2 for all the samples analyzed. The mean HER2 receptor number/core was interpolated from the standard analyzed with a linear regression fit and are shown in Table 2. All the cores showed HER2 numbers below 50,000, including various types of diseased heart tissue.

Table 2. Interpolated HER2 receptor numbers of the heart tissue microarray, using the standard shown in FIG. 2C.

TABLE 2
Interpolated HER2 receptor numbers of the heart tissue
microarray, using the standard showin in FIG. 2C.

| | | Mean |
|---|---|---|
| ID | Pathology Diagnosis | HER2 (#/cell) |
| 1 | Chronic rheumatic valvular disease with | 40000 |
| | calcification | |
| 2 | Chronic rheumatic valvular disease | 41,000 |
| 3 | Hepatocellular carcinoma embolus of cardiac atrium | 44,000 |
| 4 | Hypertrophic cardiomyopathy | 38,000 |
| 5 | Normal great arteries tissue | 37,000 |
| 6 | Normal cardiac atrium tissue | 37,000 |
| 7 | Normal myocardial tissue (focal mild hypertrophy) | 38,000 |
| 8 | Normal auricle of heart tissue | 48,000 |
| 9 | Normal myocardial tissue (mild hypertrophy) | 38,000 |
| 10 | Normal myocardial tissue | 38,000 |

The above results demonstrate the technical capabilities and potential utility of the assay disclosed herein technology using HER2 as an example. This assay can easily be extended to other ErbB family members, other cell surface targets and intracellular proteins as well. The HER2 field is a special case wherein the clinical utility of its measurement has been demonstrated, and consequently the diagnostic assay space is crowded. This assay will be used for analysis of novel HER2-directed therapeutics and the study of HER2-expressing tumor types beyond breast and gastric.

Endnotes

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein. The disclosure of each and every US, international or other patent or patent application or publication referred to herein is hereby incorporated herein by reference in its entirety.

1. A method of quantitatively measuring levels of a cellular protein in each of a plurality of cells in a section of a tissue sample so as to obtain an at least two dimensional (e.g., length and width) map of quantified density distribution of the cellular protein across the section, the method comprising:
preparing a section from a tissue sample, said section comprising identifiable cells;
staining the section with a first stain specific to the cellular protein, a second stain specific to cell nuclei, and a third stain allowing the discrimination of target cells from non-target cells, wherein the first, second and third stains are distinguishable from each other when the stained section is imaged;
obtaining one or more microscopic images of the section wherein the first, second and third stains can be discriminated
identifying target cells within the one or more images based upon staining with the second and third stains;
measuring the intensity of staining with the first stain for a plurality of the identified target cells to obtain a plurality of cellular protein staining intensity data for individual cells and recording cell location coordinate data in association with cellular protein staining intensity data for each individual cell;
ascertaining a level of stained cellular protein that is detected in each identified target cell by comparing stain intensity of the cellular protein in each identified target cell with stain intensity of the cellular protein in each of a plurality of standard cell preparations, the plurality including multiple standard cell preparations having differing known levels of expression of the cellular protein;
creating a map of quantity distribution of the cellular protein in each of the target cells within a region of the section.

2. The method of claim 1, wherein
a) the tissue sample is a tumor sample,
b) the target cells are malignant cells, and
c) the non-target cells are stromal cells.

3. The method of claim 1, wherein the cellular protein is a cell surface receptor.

4. The method of claim 3, wherein the cell surface receptor is a growth factor receptor.

5. The method of claim 4, wherein the growth factor receptor is an EGFR family receptor.

6. The method of claim 5, wherein the EGFR family receptor is HER2, HER3, or EGFR.

7. The method of claim 1, wherein the quantity distribution of the cellular protein is a continuous distribution.

8. The method of claim 1, wherein the first stain comprises an antibody specific to the cellular protein.

9. The method of claim 1, wherein the second stain is a DNA stain.

10. The method of claim 9, wherein the second stain comprises either or both of DAPI and a Hoechst® stain.

11. The method of claim 1, wherein the third stain comprises an antibody.

12. The method of claim 11, wherein the antibody comprised by the third stain is specific to a cytokeratin.

13. The method of claim 1, wherein the map is in the form of a complementary cumulative distribution.

14. The method of claim 1, wherein the identifying and measuring and ascertaining are done by automated image analysis.

15. The method of claim 1, wherein the plurality of the identified target cells comprises at least 500 cells.

16. The method of claim 1, wherein the plurality of the identified target cells comprises at least 1,000 cells.

17. The method of claim 1, wherein the plurality of the identified target cells comprises at least 2,000 cells.

18. The method of claim 1, wherein the plurality of standard cell preparations is in the form of an array of stained standard cells.

19. The method of claim 8, wherein the antibody is a labeled antibody.

20. The method of claim 8, wherein the antibody is an unlabeled antibody that is subsequently labeled with a labeled secondary antibody specific to an antibody type characteristic of the first antibody.
